(19) United States
(12) Patent Application Publication
(10) Pub. No.: US 2006/0293242 A1
(43) Pub. Date: Dec. 28, 2006

(54) Transporting of taxoid derivatives through the blood brain barrier

(76) Inventors: Jamal Temsamani, Nimes (FR); Anthony R. Rees, Saint-Chaptes (FR)

Correspondence Address: BACHMAN & LAPOINTE, P.C., 900 CHAPEL STREET, SUITE 1201, NEW HAVEN, CT, 06510, US

(21) Appl. No.: 10/490,357

(22) PCT Filed: Sep. 26, 2002
(86) PCT No.: PCT/FR02/03290

§ 371 Date: Aug. 20, 2004

(30) Foreign Application Priority Data
Sep. 27, 2001 (FR) 01/12441

(51) Int. Cl. A61K 38/10; A61K 38/08; A61K 31/704; A61K 31/525; C07K 7/08; C07K 7/06

(52) U.S. Cl. 514/14; 514/15; 514/16; 514/34; 514/251; 530/327; 530/328; 530/329

(57) ABSTRACT

Taxoid derivatives are used in the treatment of cancers, particular cancers of the central nervous system, such as brain cancers. Taxoid derivatives are transported across the blood/brain barrier (BBB). A compound is provided which consists of at least one taxoid derivative bound to at least one vector peptide capable of increasing the solubility of the derivative and advantageously allowing it to be transported across the BBB.

### BACKGROUND OF THE INVENTION

The present invention relates to the use of taxoid derivatives in the treatment of cancers, and more particularly cancers of the central nervous system, most particularly brain cancers. The invention also mainly focuses on transporting taxoid derivatives across the blood/brain barrier (BBB). Thus, a subject of the invention is a compound consisting of at least one taxoid derivative bound to at least one vector peptide capable of increasing the solubility of said derivative and advantageously of allowing it to be transported across the blood/brain barrier. The invention also relates to the preparation of these compounds and to the pharmaceutical compositions containing them, useful for the treatment of cancers, most particularly of brain cancers.

Despite undeniable progress in combating certain cancers, it has to be accepted that, overall, cancers are progressing and that they constitute, with cardiovascular diseases, a major cause of mortality. There is currently no effective treatment against certain cancers such as brain tumors. The survival time of patients suffering from glioblastoma, for example, does not exceed one year, even if chemotherapy and radiotherapy are combined with surgery. The treatment of brain tumors is limited mainly due to the blood/brain barrier, which isolates the central nervous system from the rest of the body. Consequently, most anticancer agents have to be administered at very high doses in order to reach the central nervous system, but at the cost of considerable side effects.

The discovery of new antitumor drugs constitutes one of the priorities of medical research. Advances in antitumor chemotherapy have been obtained by virtue of drugs having new chemical structure and/or a new mechanism of action, such as the anthracyclines discovered at the beginning of the 1960s and cisplatin, about ten years later. After about twenty years, the situation very fortunately again progressed with the discovery of a family of antitumor agents of natural origin: taxoids. The available drugs are Taxol® (paclitaxel) and Taxotere® (docetaxal). Taxol® and Taxotere® have a broad spectrum of clinical activity: they make a significant contribution, for the moment, in the treatment of ovarian cancers and breast cancers resistant to anthracyclines. One of the preferred targets of this chemotherapy consists of the microtubules, which form a spindle on which the chromosomes migrate at the end of cell division (mitosis). Drugs which exert their activity on this spindle are antimitotics which block mitosis and prevent the cells from multiplying. Taxol acts on the same target, but unlike vinca-alkaloids, it stabilizes this spindle and inhibits cell multiplication in another way. Unfortunately, Taxol and its analogues have the disadvantage of being very poorly water-soluble. It is therefore generally administered to patients in a solvent (Cremophor EL), which is responsible for hypersensitivity phenomena (allergy). As in most chemotherapies, taxoids, such as Taxol, cause a decrease in white blood cells and platelets. In addition to its toxicity, it has been described in the literature that Taxol does not enter the brain and is excluded by the blood/brain barrier (Heimans et al; Ann Oncol 1994, 10: 951-953). All this greatly limits the use of this antimitotic agent for the treatment of several cancers and in particular brain tumors.

Several strategies have been proposed for improving the properties of Taxol.

Hundreds of Taxol derivatives, such as those described in patents and patent applications U.S. Pat. No. 4,814,470, WO 93/02065 and WO 94/07880, have been synthesized, but the results have been disappointing. These derivatives exhibit the same solubility problems as Taxol or are less active than Taxol.

Taxol prodrugs which are metabolized in vivo so as to release Taxol have been proposed, for example in patents and patent applications U.S. Pat. No. 5,760,72 and WO 09813059. However, in most cases, the anticancer activity is decreased.

Vectors such as liposomes (U.S. Pat. No. 5,648,090) or DHA (U.S. Pat. No. 6,080,877) have been proposed for improving Taxol solubility. However, these methods give no indication regarding the passage of Taxol across the blood/brain barrier.

The applicant has demonstrated that linear peptide vectors, such as the linear peptides derived from natural peptides like protegrin and tachyplesin, transport active molecules across the BBB and improve the pharmacological properties of these molecules. The studies and results concerning these linear peptides and their use as vectors of active molecules across the blood/brain barrier have been described in French patent application no. 98/15074 filed on Nov. 30, 1998, and in French patent application no. 99/02938 filed on Nov. 26, 1999.

### SUMMARY OF THE INVENTION

A subject of the present invention is therefore compounds consisting of at least one taxoid derivative bound to at least one linear peptide capable of increasing the solubility of said derivative and of transporting it across the blood/brain barrier.

A first group of linear peptides used in the context of the compounds of the invention are those comprising a transduction domain. The term “transduction domain” is intended to mean a peptide sequence capable of penetrating into cells. By way of examples of transduction domains, and in a nonlimiting manner, mention may be made of:
Peptides derived from the HIV1 Tat protein [Fawell et al, Proc. Natl. Acad. Sci 91: 664 (1994); Schwarze et al, Science 285; 1569 (1999)]. Examples include fragment 48-60 of the tat protein of sequence SEQ ID No. 1: Gly-Arg-Lys-Lys-Arg-Arg-Gln-Arg-Arg-Arg-Pro-Pro-Gln, and a fragment comprising the sequence such as fragment 37-72.
Penetratin [Derossi et al, J. Biol. Chem 269, 10444 (1994); U.S. Pat. No. 5,888,762], of sequence SEQ ID No. 2: Arg-Gln-Ile-Lys-Ile-Trp-Pha-Gln-Asn-Arg-Arg-Met-Lys-Trp-Lys-Lys.
Signal sequences, or membrane translocation sequences (MTSs) for peptides. The sequences are recognized by a protein acceptor which participates in the addressing of the pre-protein from the translation machinery into the membrane of the appropriate intercellular organelle. MTSs which direct proteins into the same intracellular compartment, such as the endoplasmic reticulum (ER) or the mitochondria, share several structural characteristics. ER MTSs contain 17 to 52 amino acids organized with a positively charged section at the N-terminal end, a hydrophobic intersegment and a polar C-terminal region with peptidase recognition sites. Signal sequences that may be mentioned include those of sequence SEQ ID No. 3: Gly-Ala-Leu-Phe-Leu-Gly-Trp-Leu-Gly-Ala-Ala-Gly-Ser-Thr-Met-Gly-Ala-Trp-Ser-Gln-Pro-Lys-Lys-Lys-Arg-Lys-Val, and of sequence SEQ ID No. 4: Ala-Ala-Val-Ala-Leu-Leu-Pro-Ala-Val-Leu-Leu-Ala-Leu-Leu-Ala-Pro.

A second group of linear peptides according to the invention is derived from protegrins and tachyplesins. Protegrins and tachyplesins are natural antibiotic peptides, the structure of which is of the hairpin type, maintained by disulfide bridges. These bridges play an important role in the cytolytic activity observed on human cells.

The name “protegrins” refers to a set of five peptides denoted PG-1, PG-2, PG-3, PG-4 and PG-5, the sequences of which are given below, closely related and isolated from porcine leukocytes (V. N. Kokryakov et al. FEBS lett. 327, 231-236):
SEQ ID No. 5: PG-1: Arg-Gly-Gly-Arg-Leu-Cys-Tyr-Cys-Arg-Arg-Arg-Phe-Cys-Val-Cys-Val-Gly-Arg-NH₂
SEQ ID No. 6: PG-2: Arg-Gly-Gly-Arg-Leu-Cys-Tyr-Cys-Arg-Arg-Arg-Phe-Cys-Ile-Cys-Val-NH₂
SEQ ID No. 7: PG-3: Arg-Gly-Gly-Gly-Leu-Cys-Tyr-Cys-Arg-Arg-Arg-Phe-Cys-Val-Cys-Val-Gly-Arg-NH₂
SEQ ID No. 8: PG-4: Arg-Gly-Gly-Arg-Leu-Cys-Tyr-Cys-Arg-Gly-Trp-Ile-Cys-Phe-Cys-Val-Gly-Arg-NH₂
SEQ ID No. 9: PG-5: Arg-Gly-Gly-Arg-Leu-Cys-Tyr-Cys-Arg-Pro-Arg-Phe-Cys-Val-Cys-Val-Gly-Arg-NH₂

Tachyplesins (Tamura, H. et al., 1993, Chem. Pharm. Bul. Tokyo 41, 978-980), denoted T1, T2 and T3 and polyphemusins (Muta, T., 1994, CIBA Found. Sym. 186, 160-174), denoted P1 and P2, the sequences of which are given below, are homologous peptides isolated from the hemolymph of two crabs, Tachyplesus tridentatus for tachyplesins T1, T2 and T3 and Limmulus polyphemus for polyphemusins P1 and P2:
SEQ ID No. 10: P1: Arg-Arg-Trp-Cys-Phe-Arg-Val-Cys-Tyr-Arg-Gly-Phe-Cys-Tyr-Arg-Lys-Cys-Arg-NH₂
SEQ ID No. 11: P2: Arg-Arg-Trp-Cys-Phe-Arg-Val-Cys-Tyr-Lys-Gly-Phe-Cys-Tyr-Arg-Lys-Cys-Arg-NH₂.

Protegrins, tachyplesins and polyphemusins contain a high proportion of basic residues (lysines and arginines) and have four cysteines which form two parallel disulfide bridges. These three families of peptides also exhibit homologies with some defensins, and in particular with human defensin NP-1 (Kokryakov, V. N. et al., 1993, Fabs Let. 327, 231-236).

A subject of the invention is therefore a compound consisting of at least one taxoid derivative bound to at least one linear peptide chosen from the group comprising:
a linear peptide derived from protegrins or from tachyplesins,
a linear peptide comprising a transduction domain.

The invention envisions most particularly as linear peptides comprising a transduction peptide chosen, in a nonlimiting manner, from:
the transduction domains derived from the HIV1 Tat protein,
the transduction domains derived from the third helix of Antennapedia,
the transduction domains of a signal sequence.

The invention most particularly envisions as linear peptides derived from protegrins, a peptide which corresponds to formula (I) below:

BX(X or B)BXXXXBBBXXXXXXB (I)

and as linear peptide derived from tachyplesins, a peptide which corresponds to formula (II) below:

BXXXBXXXBXXXXBBXB (II)

in which:
the B groups, which may be identical or different, represent an amino acid residue whose side chain carries a basic group, and
the X groups, which may be identical or different, represent an aliphatic or aromatic amino acid residue,
or said peptides of formula (I) or (II), in retro form, consisting of amino acids in the D and/or L configuration,
or a fragment thereof consisting of a sequence of at least 5, and preferably of at least 7, successive amino acids of the peptides of formula (I) or (II).

The invention also envisions as linear peptides derived from protegrins, a peptide which corresponds to formula (I) below:

BX(X or B)BXXXXBBBXXXXXXB (I)

and as linear peptide derived from tachyplesins, a peptide which corresponds to formula (II) below:

BXXXBXXXBXXXXBBXB (II)

in which:
the B groups, which may be identical or different, are chosen from arginine, lysine, diaminoacetic acid, diaminobutyric acid, diaminopropionic acid and ornithine,
the X groups, which may be identical or different, are chosen from glycine, alanine, valine, norleucine, isoleucine, leucine, cysteine, cysteine, penicillamine, methionine, serine, threonine, asparagine, glutamine, phenylalanine, histidine, tryptophan, tyrosine, proline, aminobutyric acid, amino-1-cyclohexane carboxylic acid, aminoisobutyric acid, 2-aminotetralin carboxylic acid, 4-bromophenylalanine, tert-leucine, 4-chlorophenylalanine, beta-cyclohexylalanine, 3,4-dichlorophenylalanine, 4-fluorophenylalanine, homoleucine, beta-homoleucine, homophenylalanine, 4-methylphenylalanine, 1-naphthylalanine, 2-naphthylalanine, 4-nitrophenylalanine, 3-nitrotyrosine, norvaline, phenylglycine, 3-pyridylalanine and [2-thienyl]alanine,
or said peptides of formula (I) or (II), in retro form, consisting of amino acids in the D and/or L configuration,
or a fragment thereof consisting of a sequence of at least 5, and preferably of at least 7, successive amino acids of the peptides of formula (I) or (II).

By way of examples of taxoid derivatives used in the compounds of the invention, mention may be made of Taxol, Taxotere, or any other Taxol derivative which is substituted at one or more positions such as positions C7, C9, C10, C19, R, etc.

The bond between the taxoid derivative and the linear peptide in the compounds of the invention is chosen from a covalent bond, a hydrophobic bond, an ionic bond, and a bond which is cleavable or a bond which is not cleavable in physiological media or inside the cells.

This bond may be direct or indirect via a linker arm, and effected by means of a functional group naturally present or introduced either on or onto the peptide, or on or onto the peptide derivative, or on or onto both. This linker arm, if it is present, must be acceptable given the chemical nature and the hindrance both of the peptide and of the taxoid derivative. By way of example of such linkers containing alkyl, aryl, aralkyl or peptide groups, mention may be made of alkyl, aryl or aralkyl esters, aldehydes or acids, anhydride groups, sulfhydryl groups or carboxyl groups such as the derivatives of maleimidylbenzoic acid and of maleimidylpropionic acid and succinimidyl derivatives, groups derived from cyanogen bromide or cyanogen chloride, carbonyldiimidazole, succinimide esters or sulphonyl halides.

As functional groups, mention may be made of: —OH, —SH, —COOH or —NH₂. Thus, the taxoid derivative(s) may be bound by covalent bonds at the N-terminal or C-terminal ends or else on the side chains of the peptide.

A preferred type of bond between the taxoid derivative(s) and the linear peptide(s) involves at least one disulfide bridge. Specifically, this type of bond is characterized by its stability in the plasma after injection of the compound, and then, once the compounds of the invention have crossed the blood/brain barrier, said disulfide bridge is reduced, releasing the taxoid derivative. The bond may be effected at any site on the peptide as indicated above.

A subject of the present invention is also a method for treating cancers, particularly brain cancers, consisting in administering to an individual suffering from such a cancer an effective amount of a compound described above. The invention therefore relates to a pharmaceutical composition for treating cancers, and particularly brain cancers, comprising, as active agent, at least one compound described above.

Preferably, said pharmaceutical composition is in a form suitable for parenteral, oral, rectal, nasal, transdermal, pulmonary, central or systemic administration.

As indicated above, the linear peptides used in the context of the compounds of the invention are notable in that they are capable of solubilizing the taxoid derivatives in water and thus of allowing new formulations of these derivatives capable of increasing their effectiveness and of decreasing the side effects.

A subject of the invention is therefore most specially pharmaceutical compositions for treating cancers, and more particularly brain cancers, comprising at least one taxoid derivative, characterized in that said taxoid derivative is in the form of a water-soluble compound in which the taxoid derivative is bound to at least one linear peptide as defined above.

A subject of the invention is also the use of a linear peptide as defined above, for solubilizing a taxoid derivative.

A subject of the invention is also the use of a linear peptide defined above, for preparing a medicinal product intended for the treatment and/or for the prevention of cancers, said peptide being bound to at least one taxoid derivative so as to make said derivative water-soluble.

The invention also relates to the use of a linear peptide defined above, for preparing a medicinal product intended for the treatment and/or for the prevention of brain cancers, said peptide being bound to at least one taxoid derivative so as to transport said derivative across the BBB.

Finally, the invention relates to the use of a linear peptide for preparing a medicinal product as defined above to be administered to an individual in combination with another anticancer compound such as, in a nonlimiting manner, carmustine, doxorubicin, methotrexate, etc.

### BRIEF DESCRIPTION OF THE DRAWINGS

Other advantages and characteristics of the invention will emerge on reading the examples which follow concerning the preparation of a compound consisting of Taxol and of a linear peptide, and its activity. Reference will be made to the attached drawings in which:

FIG. 1 diagrammatically represents the chemical synthesis of a vectorized compound of Taxol.

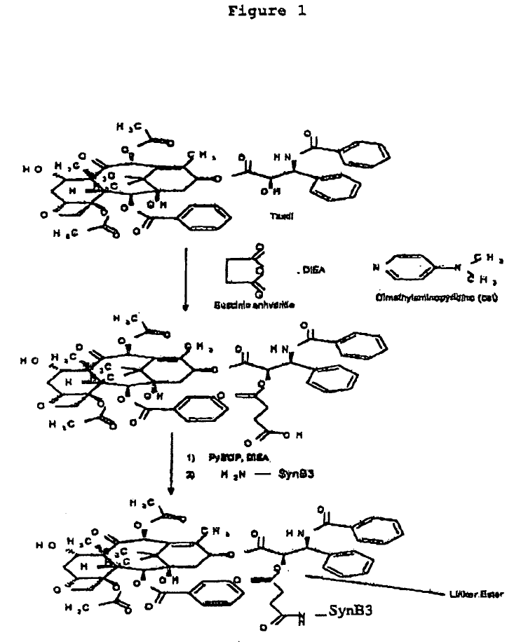

FIG. 2 illustrates the crossing of the blood/brain barrier by compound 1 (Taxol) and by compound 2 (vectorized Taxol).

### DETAILED DESCRIPTION OF THE PREFERRED EMBODIMENT(S)

I—Chemical Synthesis of Vectorized Taxol

1) Synthesis of the Vector Peptide

The peptide SynB3 of sequence SEQ ID No. 12: Arg-Arg-Leu-Ser-Tyr-Ser-Arg-Arg-Arg-Phe, is assembled on solid phase according to an Foc/tu strategy, cleaved and deprotected with trifluoroacetic acid, and then purified by reverse-phase preparative high pressure chromatography and lyophilized. Its purity (>95%) and its identity are confirmed by analytical HPLC and by mass spectrometry.

2) Coupling of Taxol to SynB3

100 mg of Taxol (paclitaxel, 1 eq) are dissolved in 1 ml of dimethylformamide (DMF). 12 mg of succinic anhydride (Succ20, 1 eq) in 120 μl of DMF are added. 1.47 mg of dimethylaminopyridine (DMAP, 0.1 eq) in 14 μl of DMF are added. 40 μl of diisopropylethylamine (DIEA, 2 eq) are added. Incubation is carried out for 1 hour. The mixture is examined for formation of paclitaxel hemisuccinate by mass spectrometry. 200 mg (1.2 eq) of the vector peptide SynB3 dissolved in 2 ml of DMF are added. 75 mg of PyBOP (1.2 eq) are added, followed by DIEA (41 μl, 2 eq). The mixture is incubated for 20 min. The mixture is examined for formation of the coupling product by mass spectrometry and analytical HPLC.

The coupling product is precipitated with ether and centrifuged and the crude is taken up in 2 ml of 50/50 H₂O/acetonitrile containing 0.1% TFA. Purification is carried out on reverse-phase preparative HPLC, followed by lyophilization.

II—Compounds Tested

The compounds tested are given in table 1 below.

TABLE 1

| Compound | |
|---|---|
| Compound 1 | Taxol |
| Compound 2 | Taxol-SynB3 |

1) Assay Used: Cytotoxicity Assay

9 L (glioblastome), MCF-7 and MDA-MB-435 (breast cancer), and SK-N-SH (neuroblastome) cells were obtained commercially from ATCC. The cells are seeded at approximately 10⁴ cells per well, 24 h before addition of the products. They are then at 60-80% confluence on the day of the experiment. The cells are maintained in culture at 37° C. in an atmosphere at 95% humidity and 5% CO₂ in an OptiMem® medium.

The cells are incubated either with free Taxol or with the vectorized Taxol at increasing concentrations for 48 hours to 72 hours. At the end of the culturing period, MTT (3-(4,5-dimethylthiazole-2-yl)-2,5-diphenyltetrazolium bromide) is added to the wells and the culture plates are then incubated for 4 hours in an incubator. The resulting crystalline deposit of formazan is then dissolved by addition of 200 μl of DMF/SDS. The optical density (OD) is measured at 550 nm (reference 630 nm) using a microplate reader.

Graphic representation of the percentages of OD of the treated wells as a function of the concentration of products makes it possible to determine the IC₅₀. This corresponds to the concentration of the product which inhibits 50% of growth.

2) In situ Brain Perfusion

Mice (20-25 g, Iffa-Credo, Arbresle, France) are anesthetized. After exposure of the common carotid, the right external carotid artery is ligated at the level of the bifurcation with the internal carotid and the common carotid is ligated between the heart and the site of implantation of the catheter (polyethylene catheter, ID: 0.76). This catheter, prefilled with a solution of heparin (100 units/ml), is inserted into the common carotid. The mice are perfused with the perfusion buffer (128 mM NaCl, 24 mM NaHCO₃, 4.2 mM KCl, 2.4 mM NaH₂PO₄, 1.5 mM CaCl₂, 0.9 mM MgSO₄ and 9 mM D-glucose) This buffer is filtered and then a mixture containing 95% 02/5% CO₂ is bubbled in in order to maintain the pH close to 7.4 and to provide the brain with oxygen during the perfusion.

The mice are perfused with the buffer containing free Taxol or vectorized Taxol. In each product, the Taxol is radiolabeled with carbon 14 (specific activity: 55 mCi/mmole, Sigma, France). Just before the beginning of perfusion, the heart is stopped by sectioning the ventricles, in order to avoid reflux of the perfusate during perfusion. The right hemisphere is then perfused at a rate of 10 ml/min for 60 seconds, after which the mouse is decapitated. The amount of radioactivity in the right hemisphere is then measured and the brain penetration (Kin) is calculated.

III—Results

1) Solubility

As indicated above, Taxol is very insoluble in water or in biological buffers. When the Taxol was vectorized with the vector peptide SynB3, its solubility was greater than 10 g/l. This indicates that the vectorization makes it possible to significantly improve the solubility of the Taxol.

2) Cytotoxicity

The sensitivity of the 9L, MCF-7, MDA-MB-435 and SK-N-SH cells to free Taxol and to the vectorized Taxol was measured by the MTT test under the experimental conditions defined above for which the relationship between the optical density and the number of viable cells is linear.

These cells are incubated with increasing concentrations of product and, after 24 hours of incubation, cell survival is measured by the MTT test.

Table 2 represents the concentrations of products resulting in 50% inhibition of growth (IC₅₀), determined for the free and vectorized Taxol.

TABLE 2
Results of cytotoxicity in the cancer cells

| | 9L | MCF-7 | MDA-MB-435 | SK-N-SH |
|---|---|---|---|---|
| Free Taxol | 3.5 μM | 0.042 μM | 0.095 μM | 0.087 μM |
| Taxol-SynB3 | 0.6 μM | 0.05 μM | 0.096 μM | 0.087 μM |

Our results show that the IC₅₀ for free Taxol is 3.5 μM, whereas that of the vectorized Taxol is 0.6 μM. This indicates that the vectorized Taxol is approximately 5 times more active than the free Taxol in the 9 L cell line. In the other cell lines, the IC₅₀ is the same for the two products.

3) Penetration Into the Brain

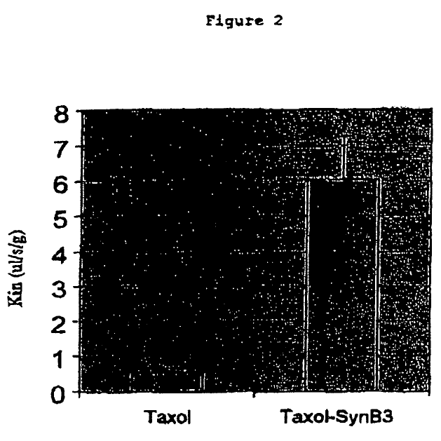

In this study, we compared the BBB penetration of free Taxol with that of the vectorized Taxol. The two products were perfused into the brain of the mouse. After perfusion for 60 seconds in the buffer, the penetration of the product is estimated by the influx constant, or Kin, in μl/sec/g. FIG. 2 shows that transporting the Taxol with the SynB3 vector increases its passage into the brain approximately 12-fold after perfusion for 60 seconds in buffer.

1-15. (canceled)

16. The use of a linear peptide chosen from the group comprising a linear peptide derived from protegrins or from tachyplesins, and a linear peptide comprising a transduction domain, for solubilizing a taxoid derivative bound to said linear peptide.

17. The use as claimed in claim 16, wherein the linear peptide is a derivative of protegrins or of tachyplesins, chosen from those of formula (I) or (II) below:

| | |
|---|---|
| BX(X or B)BXXXXBBBXXXXXXB | (I) |
| BXXXBXXXBXXXXBBXB | (II) |

in which:
the B groups, which may be identical or different, represent an amino acid residue whose side chain carries a basic group, and
the X groups, which may be identical or different, represent an aliphatic or aromatic amino acid residue, or said peptides of formula (I) or (II), in retro form, consisting of amino acids in the D and/or L configuration,
or a fragment thereof consisting of a sequence of at least 5 successive amino acids of the peptides of formula (I) or (II).

18. The use as claimed in claim 17, wherein said fragment consists of a sequence of at least 7 successive amino acids of the peptides of formula (I) or (II).

19. The use as claimed in claim 16, wherein the linear peptide is a derivative of protegrins or of tachyplesins, chosen from those of formula (I) or (II) below:

| | |
|---|---|
| BX(X or B)BXXXXBBBXXXXXXB | (I) |
| BXXXBXXXBXXXXBBXB | (II) |

in which:
B is chosen from arginine, lysine, diaminoacetic acid, diaminobutyric acid, diaminopropionic acid and ornithine,
X is chosen from glycine, alanine, valine, norleucine, isoleucine, leucine, cysteine, cysteineAcm, penicillamine, methionine, serine, threonine, asparagine, glutamine, phenylalanine, histidine, tryptophan, tyrosine, proline, Abu, amino-1-cyclohexane carboxylic acid, Aib, 2 aminotetralin carboxylic acid, 4-bromophenylalanine, tert-leucine, 4-chlorophenylalanine, beta-cyclohexylalanine, 3,4-dichlorophenylalanine, 4-fluorophenylalanine, homoleucine, beta-homoleucine, homophenylalanine, 4-methylphenylalanine, 1-naphthylalanine, 2-naphthylalanine, 4-nitrophenylalanine, 3-nitrotyrosine, norvaline, phenylglycine, 3-pyridylalanine and [2-thienyl]alanine.

20. The use as claimed in claim 16, wherein the linear peptide comprises a transduction domain chosen from the group comprising:
the transduction domains derived from the HIV1 Tat protein,
the transduction domains derived from the third helix of Antennapedia,

21. The use as claimed in claim 16, wherein the linear peptide is chosen from the group comprising the peptide of sequence SEQ ID No. 1 in the attached sequence listing, the peptide of sequence SEQ ID No. 2 in the attached sequence listing, the peptide of sequence SEQ ID No. 3 in the attached sequence listing, the peptide of sequence SEQ ID No. 4 in the attached sequence listing and the peptide of sequence SEQ ID No. 12 in the attached sequence listing.

22. The use as claimed in claim 16, wherein the bond between the taxoid derivative and the linear peptide is chosen from a covalent bond, a hydrophobic bond, an ionic bond, and a bond which is cleavable or a bond which is not cleavable in physiological media or inside the cells.

23. The use as claimed in claim 16, wherein the bond between the taxoid derivative and the linear peptide is a bond which is direct or indirect via a linker arm.

24. The use as claimed in claim 16, wherein the bond between the taxoid derivative and the linear peptide is effected by means of a functional group naturally present or introduced either on or onto the peptide, or on or onto the taxoid derivative, or on or onto both.

25. The use as claimed in claim 16, wherein the taxoid derivative is bound by covalent bonds at the N-terminal or C-terminal ends or else on the side chains of the peptide.

26. The use as claimed in claim 16, wherein the taxoid derivative is chosen from Taxol, Taxotere, or any other Taxol derivative which is substituted at at least one position.

27. The use as claimed in claim 26, wherein said at least one position is selected from the group consisting of positions C7, C9, C10, C19 and R.

28. A pharmaceutical composition for treating cancers, comprising at least one taxoid derivative bound to at least one linear peptide as claimed in claim 16, wherein said composition is substantially free of solvent.


SEQUENCE LISTING (12 sequences)

SEQ ID NO: 1 - protein, 13 aa, Human immunodeficiency virus type 1
  PEPTIDE (1)..(13): Fragment 48-60 of HIV1 Tat protein
GRKKRRQRRRPPQ

SEQ ID NO: 2 - protein, 16 aa, Drosophila melanogaster
  PEPTIDE (1)..(16): Penetratin sequence
RQIKIWFQNRRMKWKK

SEQ ID NO: 3 - protein, 27 aa, Unknown
  PEPTIDE (1)..(27): Chimera of the gp41 viral protein hydrophobic terminal domain and of the SV40 antigen nuclear localisation signal
GALFLGWLGAAGSTMGAWSQPKKKRKV

SEQ ID NO: 4 - protein, 16 aa, Unknown
  PEPTIDE (1)..(16): Peptide derived from the hydrophobic domains of K-FGF signal sequences
AAVALLPAVLLALLAP

SEQ ID NO: 5 - protein, 18 aa, Sus sp.
  PEPTIDE (1)..(18): Protegrin PG-1
RGGRLCYCRRRFCVCVGR

SEQ ID NO: 6 - protein, 16 aa, Sus sp.
  PEPTIDE (1)..(16): Protegrin PG-2
RGGRLCYCRRRFCICV

SEQ ID NO: 7 - protein, 18 aa, Sus sp.
  PEPTIDE (1)..(18): Protegrin PG-3
RGGGLCYCRRRFCVCVGR

SEQ ID NO: 8 - protein, 18 aa, Sus sp.
  PEPTIDE (1)..(18): Protegrin PG-4
RGGRLCYCRGWICFCVGR

SEQ ID NO: 9 - protein, 18 aa, Sus sp.
  PEPTIDE (1)..(18): Protegrin PG-5
RGGRLCYCRPRFCVCVGR

SEQ ID NO: 10 - protein, 18 aa, Limulus polyphemus
  PEPTIDE (1)..(18): Polyphemusin P1
RRWCFRVCYRGFCYRKCR

SEQ ID NO: 11 - protein, 18 aa, Limulus polyphemus
  PEPTIDE (1)..(18): Polyphemusin P2
RRWCFRVCYKGFCYRKCR

SEQ ID NO: 12 - protein, 10 aa, Unknown
  PEPTIDE (1)..(10): SynB3
RRLSYSRRRF